(19) United States
(12) Patent Application Publication
(10) Pub. No.: US 2019/0079582 A1
(43) Pub. Date: Mar. 14, 2019

(54) APPARATUS AND METHOD PROVIDING A WEARABLE USER INTERFACE DEVICE

(71) Applicant: Thomson Licensing, Issy-les-Moulineaux (FR)

(72) Inventors: Kent Lyons, Mountain View, CA (US); Cindy Kao, Cambridge, MA (US)

(21) Appl. No.: 16/124,325

(22) Filed: Sep. 7, 2018

Related U.S. Application Data
(60) Provisional application No. 62/555,669, filed on Sep. 8, 2017.

(51) Int. Cl. G06F 3/01; D02G 3/44; B32B 7/08; B32B 7/12; B32B 3/08; B32B 27/36; B32B 37/18; B32B 37/12; B32B 38/00; D05B 3/12; B32B 27/28; B32B 27/08; A61B 5/053; A61B 5/00

(52) U.S. Cl. CPC G06F 3/014; G06F 2203/011; B32B 7/08; B32B 7/12; B32B 3/08; B32B 27/36; B32B 37/182; B32B 37/12; B32B 38/00; D05B 3/12; B32B 27/283; B32B 27/08; A61B 5/0531; G06F 3/017; G06F 3/015; A61B 5/6826; A61B 5/68335; D10B 2331/04; D10B 2321/06; D10B 2101/20; D10B 2401/16; B32B 2307/546; B32B 2367/00; B32B 2457/00; D05D 2303/40; B32B 2307/412; B32B 2383/00; B32B 2255/10; B32B 2038/008; B32B 2250/02; B32B 2250/24; A61B 2562/0219; D02G 3/441

(57) ABSTRACT

A flexible substrate including a conductive path formed on a side of the flexible substrate from a flexible conductive thread attached to the substrate and an electronic component attached to the same side of the substrate as the conductive path and electrically connected to the conductive path and having an adhesive on the same side suitable for adhering the flexible substrate including the conductive path and the electronic component on a region of skin of a user's body.

### CROSS REFERENCE TO RELATED APPLICATION

This application claims the benefit under 37 U.S.C. § 119 of Provisional Application 62/555,669 filed on Sep. 8, 2017.

### TECHNICAL FIELD

The present principles relate generally to wearable devices for user interface applications and methods for creating such devices.

### BACKGROUND

High-fidelity wearable devices to support user interface applications such as for tracking systems, e.g., for finger-level hand tracking such as for hand gesture tracking, offer enormous potential to expand the input modalities of ubiquitous computing systems for applications such as context-aware sensing and virtual-reality. Optical-based systems are known to those skilled in the art and provide techniques which offer high-accuracy. However, such systems require specific hardware and line-of-sight view for operation. Such requirements may not be compatible with certain real-world applications. Wearable systems placing sensors directly on the hand may be able to overcome these constraints. However, to accurately track finger movement, systems often come in a glove form factor to enhance sensing coverage. Acceptance of the glove form factor may be problematic for a user due to issues around convenience, comfort, and social perception.

Systems involving on-skin interfaces for user interactions are known to those skilled in the art of human/computer interaction (HCI) and wearable technology. However, existing on-skin user interface approaches may involve on-skin fabrication requiring highly-specialized and costly manufacturing techniques. In addition, such approaches may provide only limited wiring complexity and require external components such as an external rigid microcontroller component and/or a power source.

### SUMMARY

These and other drawbacks and disadvantages of the prior art are addressed by the present principles, which are directed to wearable devices such as wearable tracking devices such as for hand-gesture tracking.

According to an aspect of the present principles, there is provided in an exemplary embodiment a tracking system in a self-contained form factor configured for on-skin use that includes a plurality of sensors, conductive connections and electronic components.

In accordance with another aspect, an embodiment of a method to create an on-skin tracking system includes depositing conformal multi-stranded metallic wires on a thin silicone substrate.

In accordance with another aspect, an embodiment of a method to create an on-skin tracking system includes sewing conductors to a silicone substrate using a sewing machine, thereby enabling grouping of multiple wires to decrease wiring complexity.

In accordance with another aspect, in an embodiment of a method to create an on-skin tracking system, rigid electronic components are positioned in separate regions or islands configured for distribution throughout a region of skin to which the tracking system is affixed.

According to another aspect of the present principles, there is provided in an exemplary embodiment a method comprising fastening a sheet of flexible material on a first side of a flexible substrate layer; adhering a flexible conductive thread to a second side of the flexible substrate; fastening an electronic component to the substrate and to the conductive path at the location to form an electrical connection of the component to the conductive path; and applying an adhesive to the second side of the substrate, thereby enabling affixing the substrate including the conductive path and the electronic component to a region of skin of a user's body.

According to another aspect of the present principles, the sheet of flexible material provides a pattern for fastening the flexible conductive thread to the second side of the flexible substrate.

According to another aspect of the present principles, there is provided in an exemplary embodiment a method comprising fastening a sheet of flexible material on a first side of a flexible substrate layer, wherein a pattern on the flexible material indicates a conductive path and a location of an electronic component; adhering a flexible conductive thread to a second side of the flexible substrate and following the pattern of the conductive path on the sheet of flexible material to form the conductive path; fastening the electronic component to the substrate and to the conductive path at the location to form an electrical connection of the component to the conductive path; and applying an adhesive to the second side of the substrate, thereby enabling affixing the substrate including the conductive path and the electronic component to a region of skin of a user's body.

In accordance with another aspect of the present principles, the preceding exemplary embodiment of a method further comprises forming the pattern on the sheet of flexible material prior to fastening the sheet of flexible material to the first side of the flexible substrate layer.

In accordance with another aspect of the present principles, an exemplary embodiment of a method comprises forming a circuit pattern on a flexible transparent sheet of PET material, wherein the pattern indicates a conductive circuit path and a location of an electronic component; fastening the transparent sheet of PET material on a first side of a flexible silicone substrate layer; adhering a flexible conductive thread to a second side of the flexible silicone substrate layer and along the conductive circuit path of the pattern on the flexible transparent sheet of PET material to form the conductive circuit path; soldering the electronic component to the conductive thread at the location to electrically couple the electronic component to the conductive circuit path; and applying an adhesive to the second side of the flexible silicone substrate layer to enable affixing the flexible silicone substrate layer including the conductive circuit path and electronic component to a region of skin of a user's body to form an on-body printed circuit board (PCB).

In accordance with another aspect of the present principles, any of the preceding exemplary embodiments of a method may further comprise adhering the conductive thread to the second side of the substrate by sewing the conductive thread to the second side of the substrate.

In accordance with another aspect of the present principles, any of the preceding exemplary embodiments of a method including sewing may further comprise stitching along the pattern using a sewing machine having an upper thread and a lower thread wherein the lower thread comprises the flexible conductive thread, and stitches created by the stitching fasten the sheet of flexible material on the first side of the flexible substrate using the first thread and adhere the flexible conductive thread to the second side of the flexible substrate.

In accordance with another aspect of the present principles, any of the preceding exemplary embodiments of a method may include following applying adhesive by, in the order named: adhering the second side of the substrate to the skin of a user using the adhesive; dissolving the upper thread fastening the flexible sheet of material to the substrate; and removing the flexible sheet of material from the substrate while leaving the substrate adhered to the skin of the user.

In accordance with another aspect of the present principles, an exemplary embodiment of apparatus comprises: a sheet of flexible material; a flexible substrate layer having a first side to which the sheet of flexible material is fastened; a flexible conductive thread adhered to a second side of the flexible substrate to form the conductive path, wherein the electronic component is fastened to the flexible substrate layer and to the conductive path at the location to form an electrical connection of the component to the conductive path; and an adhesive on the second side of the flexible substrate layer configured to enable affixing the flexible substrate layer including the conductive path and the electronic component to a region of skin of a user's body.

According to another aspect of the present principles, the sheet of flexible material provides a pattern for fastening the flexible conductive thread to the second side of the flexible substrate.

In accordance with another aspect of the present principles, an exemplary embodiment of apparatus comprises: a sheet of flexible material having a pattern thereon indicating a conductive path and a location of an electronic component; a flexible substrate layer having a first side to which the sheet of flexible material is fastened; a flexible conductive thread adhered to a second side of the flexible substrate and following the pattern of the conductive path on the sheet of flexible material to form the conductive path, wherein the electronic component is fastened to the flexible substrate layer and to the conductive path at the location to form an electrical connection of the component to the conductive path; and an adhesive on the second side of the flexible substrate layer configured to enable affixing the flexible substrate layer including the conductive path and the electronic component to a region of skin of a user's body.

In accordance with another aspect of the present principles, another exemplary embodiment of apparatus comprises: a sheet of flexible transparent PET material having a pattern thereon indicating a conductive circuit path and a location of an electronic component; a flexible silicone substrate layer having a first side to which the sheet of flexible transparent PET material is fastened; a flexible conductive thread adhered to a second side of the flexible silicone substrate material and following the pattern of the conductive circuit path on the sheet of flexible transparent PET material to form the conductive circuit path, wherein the electronic component is fastened to the flexible silicone substrate layer and to the conductive circuit path at the location to electrically couple the electronic component to the conductive circuit path; and an adhesive on the second side of the flexible silicone substrate layer to enable affixing the flexible silicone substrate layer including the conductive circuit path and the electronic component to a region of skin of a user's body to form an on-body printed circuit board (PCB).

In accordance with another aspect of the present principles, any of the preceding exemplary embodiments of apparatus may comprise adhering the flexible conductive thread to the second side of the flexible substrate layer by sewing the conductive thread to the second side of the flexible substrate layer.

In accordance with another aspect of the present principles, any of the preceding exemplary embodiments of apparatus including sewing may further comprise stitching along the pattern using a sewing machine having an upper thread and a lower thread wherein the lower thread comprises the flexible conductive thread, and stitches created by the sewing machine fasten the sheet of flexible material on the first side of the flexible substrate layer using the first thread and adhere the flexible conductive thread to the second side of the flexible substrate layer.

In accordance with another aspect of the present principles, any of the preceding exemplary embodiments of apparatus may further comprise the flexible substrate layer being affixed to the skin of a user using the adhesive and thereafter the upper thread being dissolved enabling removal of the flexible sheet of material.

In accordance with another aspect of the present principles, any of the preceding exemplary embodiments of apparatus may further comprise the electronic component including a sensor affixed to the location on the flexible substrate layer and wherein the substrate including the sensor is configured for attachment to a hand of a user such that the sensor will be positioned on a digit of the hand.

In accordance with another aspect of the present principles, any of the preceding exemplary embodiments of apparatus wherein the electronic component comprises a sensor may further comprise the sensor including at least one inertial measurement unit (IMU) and further comprise a processor fastened to a second location on the flexible substrate layer, wherein the IMU is coupled to the processor by the conductive path to enable the processor to process signals produced by the sensor to track a movement of the hand when the flexible substrate layer including the IMU and processor is attached to the hand.

In accordance with another aspect of the present principles, any of the preceding exemplary embodiments of apparatus including an IMU may further comprise the at least one IMU including four IMUs positioned at respective locations on the flexible substrate layer selected to place two IMUs on each of the index finger digit and thumb digit of the hand when the flexible substrate layer is attached to the hand and the conductive path comprises a plurality of conductive paths coupling each IMU to the processor by respective ones of the plurality of conductive paths wherein the processor processes signals produced by the four IMUs to track a movement of the finger digit relative to the thumb digit of the hand.

In accordance with another aspect of the present principles, any of the preceding exemplary embodiments of apparatus may comprise a wearable device.

In accordance with another aspect of the present principles, any of the preceding exemplary embodiments of apparatus may further comprise the electronic component including at least one sensor for sensing a characteristic of the user.

In accordance with another aspect of the present principles, any of the preceding exemplary embodiments may further comprise one or more of the following features:
the flexible conductive thread comprises a conductive braided wire;
the upper thread comprises a thread formed from dissolvable PVA material;
the adhesive comprises a tattoo paper adhesive;
the sheet of flexible material comprises a transparent PET material and the flexible substrate comprises a silicone material.

In accordance with another aspect of the present principles, any of the preceding exemplary embodiments including the pattern may further comprise the pattern providing a guide for fastening the flexible conductive thread to form the conductive path, and the pattern comprising at least one of a marking on a surface of the sheet of flexible material, an edge of the sheet of flexible material, a projected pattern projected on to a surface of the flexible sheet of material, and a freeform path determined during the fastening as the fastening proceeds.

In accordance with another aspect of the present principles, any of the preceding exemplary embodiments wherein the flexible conductive thread comprises braided metallic wire may include bundling or grouping multiple traces into one conductive path.

### BRIEF DESCRIPTION OF THE DRAWING

The present principles may be better understood in accordance with the following exemplary figures, in which:

FIG. 1 is a diagram showing an exemplary apparatus in accordance with the present principles;

FIG. 2 is a diagram showing various exemplary embodiments of aspects of the present principles;

FIG. 3 is an illustration of an exemplary embodiment of apparatus in accordance with the present principles;

FIG. 4 is a diagram in flowchart form of an exemplary embodiment of a method in accordance with the present principles;

FIG. 5 is a diagram in flowchart form of another exemplary embodiment of a method in accordance with the present principles; and

FIG. 6 is a diagram showing an exemplary embodiment of aspects of the present principles.

In the various figures, like-numbered reference designators refer to the same or similar features.

### DETAILED DESCRIPTION

The present principles are generally directed to wearable devices and methods for creating such devices.

While one of ordinary skill in the art will readily contemplate various applications to which the present principles can be applied, the following description will focus on embodiments of the present principles applied to wearable devices for user interface applications such as tracking systems and/or monitoring characteristics of a user, e.g., applications such as on-skin finger-level hand tracking to facilitate a user interface or user interaction with various devices and systems. Such devices and systems include but are not limted to user interface applications for set-top boxes, gateway devices, digital television (DTV) devices, mobile devices such as smart phones and tablets, gaming platforms, computer-aided design (CAD) systems, virtual or augmented reality systems, etc. However, one of ordinary skill in the art will readily contemplate other devices and applications to which the present principles can be applied, given the teachings of the present principles provided herein, while maintaining the spirit of the present principles. For example, the present principles may be useful for interaction with any device that has data processing capability and accepts input from a user. It is to be appreciated that the preceding listing of devices is merely illustrative and not exhaustive.

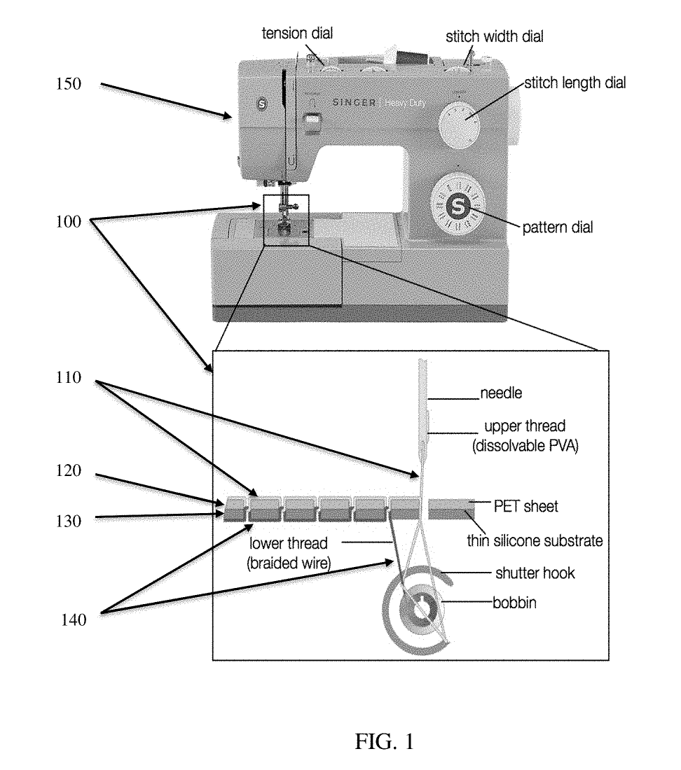

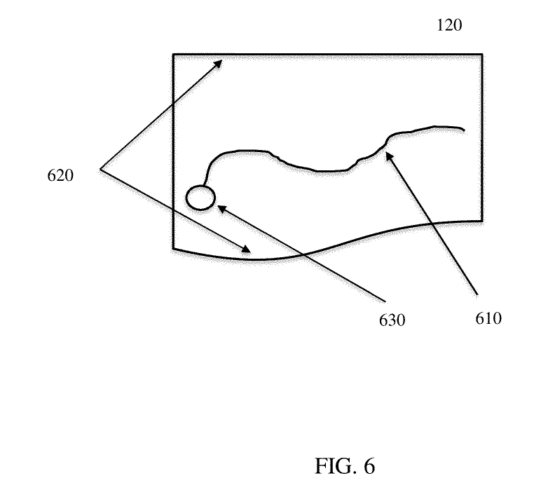

In the drawings, FIG. 1 illustrates an exemplary embodiment of apparatus in accordance with the present principles. In FIG. 1, feature 100 illustrates apparatus including a flexible sheet of material 120 fastened to a first side of a flexible substrate layer 130, e.g., an upper side of layer 130 in FIG. 1. Material 120 may be, for example, a sheet of transparent material such as polyethylene terephthalate also known as PET plastic or PETE plastic that is relatively thin such that material 120 is flexible. Layer 130 may be, for example, a layer of non-conductive material such as silicone also being relatively thin such that layer 130 is flexible. Material 120 may have a pattern on it indicating a conductive path and a location of an electronic component. As explained in more detail below in regard to FIG. 6, a pattern is not necessary but may be provided on the sheet of flexible material in any one or more of various ways. A flexible conductive thread 140 is fastened to a second side of the substrate layer 130, e.g., a lower side of substrate 130 in FIG. 1, following the pattern on material 120 to create the conductive path on the second side of layer 130. That is, the pattern if it exists on material 120 provides a track or path which the fastening of conductive thread 140 may follow in accordance with the present principles to produce the conductive path. The conductive thread may be, for example, a braided metallic wire. The substrate layer 130 may be very thin material such as silicone such that when placed on a skin region of a user as described herein the substrate does not restrict or interfere with movements of the user. As a result, the substrate layer may have limited or little structural strength. The sheet of flexible material 120 when adhered or fastened to the substrate layer helps to stabilize and/or add structural strength to the substrate layer during the fastening of the conductive flexible thread in accordance with the present principles.

In the exemplary embodiment shown in FIG. 1, a sewing machine 150 such as, for example, a Singer Heavy Duty sewing machine model 4423 may provide the described conductive thread and fastening. In more detail, a sewing machine such as 150 in FIG. 1 has features illustrated in FIG. 1 including a needle threaded with an upper thread, a bobbin filled with a lower thread and a shutter hook that, during operation of the sewing machine, intertwines the upper and lower threads to form stitches through two overlapping pieces of material to hold the pieces of material together as is well known. In the exemplary embodiment of FIG. 1, the upper thread may be a flexible thread such as poly vinyl alcohol, or PVA, thread that is also know to have the property of being dissolvable in a solvent such as water, i.e., water soluble. The lower thread in the bobbin may be a braided wire. Material 120 is placed on top of substrate material 130, i.e., on a first surface of substrate material 130, and a pattern indicating a conductive path may be included on material 120. The superimposed layers of material 120 and substrate 130 are placed on the table of sewing machine 150 between the needle and the bobbin of the sewing machine. Operation of machine 150 produces a stitching action. The stitching action forms stitches from the upper and lower threads that fasten material 120 to the upper surface of substrate 130. In addition, the interaction of the upper thread and lower conductive thread adheres the lower conductive thread to the second, or lower, side of substrate 130. The stitching action may follow a predetermined pattern, e.g., a pattern appearing on a surface of material 120, by guiding the material through machine 150 along the pattern. Or, the material may be guided through the machine freeform along a desired path to create stitches along the path. The stitching adhering the lower conductive thread to the second side of substrate layer 130 produces a conductive path through the conductive thread and along the path of the stitching, e.g., along the pattern on material 120.

Following creation of the conductive path by fastening the conductive thread 140 to the second side of substrate 130 as described above, an electronic component, e.g., a sensor or processor, may be fastened to the second side of the substrate and to the conductive path, e.g., by soldering, to form an electrical connection of the electronic component to the conductive path. Then, an adhesive may be applied to the second side of the substrate. An example of an adhesive suitable for use in accordance with the present principles comprises a tattoo paper adhesive. The combination of material 120, substrate 130, conductive path formed from thread 140, the electronic component fastened to the substrate and the adhesive form an apparatus configured to be adhered to a region of skin of a user's body using the adhesive.

In an embodiment, after the apparatus is attached to a user's body, the upper thread 110 may be dissolved, e.g., by applying water to a PVA thread in an embodiment, enabling removal of material 120 while substrate 130 remains affixed to the user's body by the adhesive. Following removal of material 120, the conductive path and electronic component remain intact, electrically connected and fixed in place between the substrate and the skin of the user's body. Thus, the apparatus in effect forms an on-body printed circuit board (PCB).

In accordance with other aspects of the present principles, a machine such as 150 in FIG. 1 can be adjusted for parameters including thread tension, stitch width, stitch length, and pattern design, using controls illustrated in FIG. 1. Adjusting these controls influence the resulting stitch. As described above, the shutter hook rotates during operation of machine 150, capturing the upper thread from the needle above, and looping it around the lower thread spooling from the bobbin below. The two threads interlock around the layers of material 120 and 130, binding them together.

Adjusting the tension of machine 150, e.g., using the tension dial, determines the vertical movement of the needle (threaded with the upper thread), and its penetration into the materials being stitched. With high tension, the upper thread stays on the upper side of the substrate. With low tension, the upper thread penetrates through the substrate and appears on both sides. The stitch width, stitch length, and pattern design dial control the horizontal movement of the needle, and enable various sewing patterns. In an embodiment of the present principles as described herein, two different threads may be used for the upper thread, e.g., dissolvable PVA, and the lower thread, e.g., conductive braided wire. In accordance with another aspect, a tension of the stitching is adjusted so the two threads remain on separate sides of the substrate, i.e., the interlocking portion of the two threads in each stitch is formed or positioned within the combined layers of material 120 and substrate 130 as shown in FIG. 1.

In an exemplary embodiment, the material 120 may comprise a material such as temporary tattoo paper, e.g., Silhouette Temporary Tattoo Paper, and substrate 130 may be a material such as a thin embroidery stabilizer. The conductive thread, e.g., upper thread in FIG. 1, may be a braided wire such as 38 AWG Remington Industries Magnet wire. The lower thread may be, e.g., a PVA thread such as YLI Wash-A-Way Thread .Although not necessary, such examples of materials 120 and 130 may be held together during creation of the conductive path, e.g., during stitching by machine 150, by an adhesive such as a temporary tattoo adhesive.

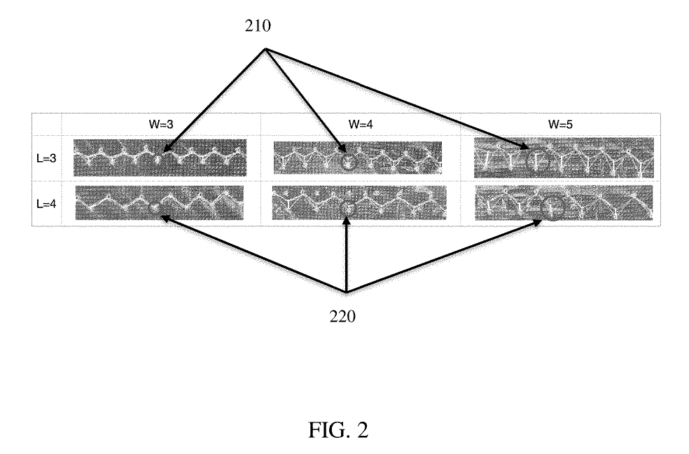

In accordance with another aspect of the present principles, fastening materials as described using a machine such as 150 in FIG. 1 enables varying certain parameters. For example, the sewing-enabled fabrication method enables the adjustment of parameters in terms of stitch tension (T), width (W), and length (L) through the sewing machine as mentioned above. Also as mentioned above, in an embodiment a stitch tension may selected to maintain the flexible conductive thread, e.g., braided wire, on one side of the substrate. The stitch width (W) and length (L) to be two parameters to alter. FIG. 2 illustrates the effect of varying W and L. In FIG. 2, images 210 illustrate stitches with three values of W, i.e., 3, 4 and 5, for a value of L of 3. Also in FIG. 2, images 220 illustrate stitches with the same three values of W as in 210 except having a value of L of 4. As shown in FIG. 2, a larger value of L generates stitches that are longer and have smaller zigzag angles. A larger value of W increases the length of the upper thread, e.g., white PVA thread in FIG. 2, that holds the lower thread, e.g., copper-color braided wire in FIG. 2, to the substrate layer. This increased length in the upper thread portion of each stitch is highlighted by the red circles in FIG. 2. It should be noted that the stitches shown in FIG. 2 are formed in the overlapping combination of material 120 and substrate 130 as shown in FIG. 1 and are transparent materials in FIG. 2, thus making it possible to see both the upper and lower thread components of the stitches in FIG. 2.

Varying parameters L and W as described herein may affect durability of the described apparatus when fastened on a region of skin of a user's body. For example, a larger value of L may increase durability. A larger value of L for stitches created by a machine such as machine 150 in FIG. 1 may result in a smaller zigzag angle of the stitch and allow for more expansion along the wire when stretched, e.g., by movement of the part of the body of a user where the apparatus is mounted such as a digit of a hand, thus increasing durability. For fixed L, a smaller W may provide increased durability because for a larger W, the upper thread may pull the edge of the lower conductive thread and increase the likelihood of breakage of the conductive thread. In an embodiment, a smaller value of W, e.g., W=3, combined with a larger value of L, e.g., L=4 may provide suitable durability for apparatus mounted on-skin as described herein.

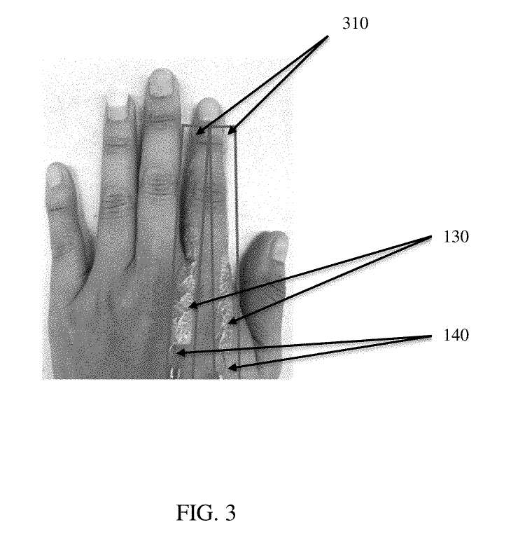

FIG. 3 illustrates an embodiment of apparatus in accordance with the present principles mounted on a region of skin of a hand. In FIG. 3, material 120 as in FIG. 1 has been removed, e.g., by dissolving in water. Substrate 130 is shown configured to extend along both sides of a digit of the hand, e.g., the index finger. A conductive path 140 formed, e.g., from the lower conductive thread during stitching is shown passing under substrate 130. One or more electronic components may be fastened to the underside of substrate 130 and coupled to conductive path 140 and positioned on the substrate to be held, e.g., at location 310 in FIG. 3.

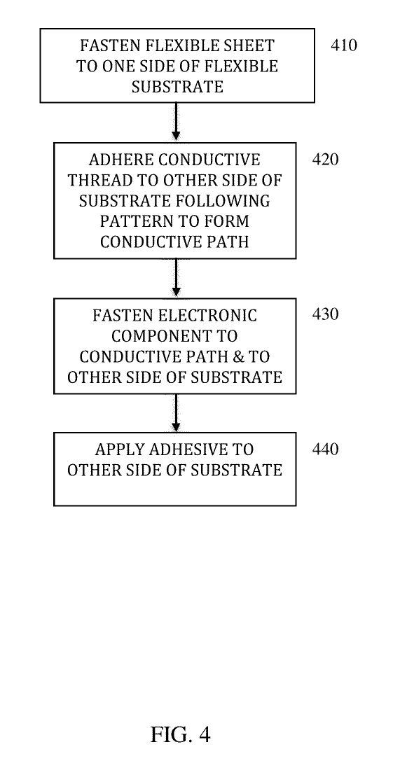

FIG. 4 shows an exemplary embodiment of a method in accordance with the present principles. In FIG. 4, at 410 a flexible sheet of material is fastened to one side of a flexible substrate. There may be a pattern on the sheet of flexible material, e.g., to form a guide for the fastening. As described in regard to the exemplary embodiment of FIG. 1, the fastening may occur by stitching involving an upper thread and a lower thread. At 420, a conductive thread is adhered to a second side of the substrate layer to form a conductive path, e.g., adhering by forming stitches as described above using a conductive thread as a lower thread component of the stitches. At 430, an electronic component is fastened to the conductive path formed at 420, e.g., by soldering. At 440, an adhesive is applied to the other or second side of the substrate.

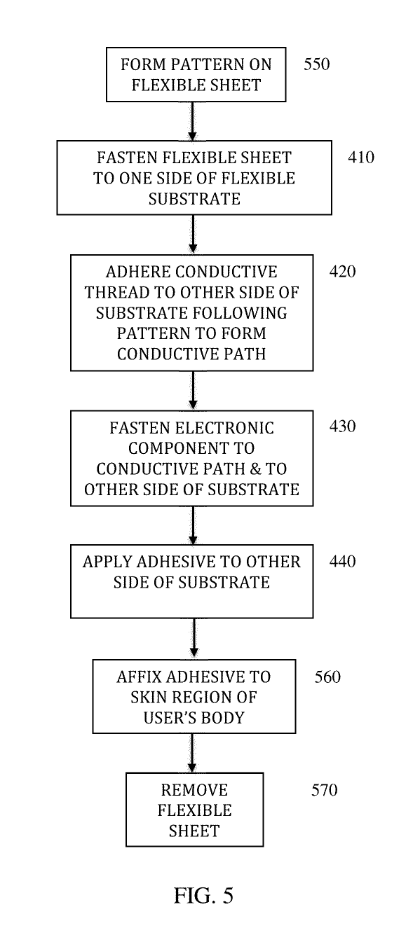

FIG. 5 shows another exemplary embodiment of a method in accordance with the present principles. The embodiment shown in FIG. 5 includes features similar to those of the embodiment in FIG. 4 as indicated by reference numbers for certain features in FIG. 5 being the same in those in FIG. 4, i.e., 410, 420, 430 and 440, and these features will not be explained again in regard to FIG. 5. Also in FIG. 5, 410 is preceded by 550 where a pattern is formed on the flexible sheet. The pattern indicates, e.g., a conductive path and a location for an electronic component, as explained herein. Feature 440 in FIG. 5 is followed by 560 where the surface of the substrate on which the adhesive was applied at 440 is applied or affixed to a region of skin of a user's body and held there by the adhesive. Then, at 570, the flexible sheet is removed, e.g., by dissolving a PVA thread component of stitches created as described herein.

FIG. 6 illustrates a thin flexible material such as material 120 in FIG. 1 having a pattern. In accordance with the present principles, a pattern is not necessary but if used with material 120 may be provided in one or more of various ways. For example, FIG. 6 illustrates thin flexible material 120 having a pattern 610 marked or drawn on the material, e.g., printed on the surface or marked by hand with a marking pen. Pattern 610 indicates the conductive path and the flexible conductive thread may be attached along or following pattern 610. For example, stitching produced during sewing as described herein may follow pattern 610. Alternatively, an edge of material 120 such as one or more edges 620 in FIG. 6 may provide a pattern. For example, stitching may occur by following an edge either freehand or by a guiding device (e.g., mechanical or optical). Also, a pattern may be projected on to the surface flexible sheet of material, e.g., by a separate projector or a projector attached to a stitching device such as sewing machine described herein. Also, as mentioned, a pattern is not necessary. That is, attachment of the flexible conductive thread such as by stitching with a sewing machine may occur freehand or freeform without a pattern. Also in FIG. 6, a location for an electronic component is indicated by 630. That is, a pattern may or may not include an indication such as 630 for a location where an electronic component is to be attached to the substrate.

In accordance with an aspect of the present principles, an embodiment including a braided metallic wire includes a plurality of conductive traces bundled or grouped together into the braided metallic wire. Fastening the braided wire to the substrate layer fastens the bundle of the plurality of traces to the substrate layer along the conductive path. Having a bundle of traces along a single conductive path on the substrate aids in controlling or reducing the complexity of wiring when multiple electronic components, e.g., multiple sensors, must be placed and electrically connected on a limited area such as a hand.

In accordance with present principles, an embodiment may comprise an electronic component affixed to a location of the flexible substrate such as a sensor for sensing a characteristic of the user. Examples of such sensors and the associated characteristics being monitored or sensed include sensors for detecting electrodermal activity (EDA) or for measuring skin conductance which may be suitable for montoring a characteristic such as an emotional state of a user. Monitoring a characteristic such as an emotional state may be useful for applications such as determining audience reaction to content experienced (e.g., viewing a movie), determining health or condition of a user, and various other applications as will be apparent to one skilled in the art. A sensor for sensing a characteristic may be one of a plurality of sensors such as a plurality of sensors attached to a flexible substrate in accordance with aspects of the present principles. A plurality of sensors may be located in various locations on the flexible substrate to form an array of sensors. All of the plurality of sensors in the array may be coupled to a processor, e.g., also mounted on the flexible substrate, by conductive paths on the flexible substrate. The processor may process one or more of the plurality of signals produced by the array of sensors for applications such as tracking movement and determining a characteristic of a user. Also, an array of sensors may include various types of sensors to provide for a combination of applications such as an inertial measurement unit (IMU) for tracking motion and an EDA sensor for determining a characteristic of a user such as emotion. Many other potential applications, types of sensors and configurations of sensors will be readily apparent to one skilled in the art based on the teachings of the present description.

The present description illustrates the present principles. It will thus be appreciated that those skilled in the art will be able to devise various arrangements that, although not explicitly described or shown herein, embody the present principles and are included within its spirit and scope. For example, various types and configurations of sensors are envisioned as described above. Also, various interpretations of terminology used herein are envisioned. For example, the term thread as used herein is intended to broadly encompass various types and forms of thin, flexible strands of material having various cross sections such as round, flat, or oval and having various dimensions that may be constant or vary along a conductive path in accordance with the present principles. Also, although attachment of conductive thread to a flexible substrate by sewing is described herein in regard to an embodiment, other methods of attachment and other methods of sewing other than the exemplary embodiment of a sewing machine as described herein are possible and envisioned.

All examples and conditional language recited herein are intended for pedagogical purposes to aid the reader in understanding the present principles and the concepts contributed by the inventor(s) to furthering the art, and are to be construed as being without limitation to such specifically recited examples and conditions.

Moreover, all statements herein reciting principles, aspects, and embodiments of the present principles, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein represent conceptual views of illustrative circuitry embodying the present principles. Similarly, it will be appreciated that any flow charts, flow diagrams, state transition diagrams, pseudocode, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

Herein, the phrase “coupled” is defined to mean directly connected to or indirectly connected with through one or more intermediate components. Such intermediate components may include both hardware and software based components.

In the claims hereof, any element expressed as a means for performing a specified function is intended to encompass any way of performing that function including, for example, a) a combination of circuit elements that performs that function or b) software in any form, including, therefore, firmware, microcode or the like, combined with appropriate circuitry for executing that software to perform the function. The present principles as defined by such claims reside in the fact that the functionalities provided by the various recited means are combined and brought together in the manner which the claims call for. It is thus regarded that any means that can provide those functionalities are equivalent to those shown herein.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

It is to be understood that the teachings of the present principles may be implemented in various forms of hardware, software, firmware, special purpose processors, or combinations thereof.

Most preferably, the teachings of the present principles are implemented as a combination of hardware and software. Moreover, the software may be implemented as an application program tangibly embodied on a program storage unit. The application program may be uploaded to, and executed by, a machine comprising any suitable architecture. Preferably, the machine is implemented on a computer platform having hardware such as one or more central processing units (“CPU”), a random access memory (“RAM”), and input/output (“I/O”) interfaces. The computer platform may also include an operating system and microinstruction code. The various processes and functions described herein may be either part of the microinstruction code or part of the application program, or any combination thereof, which may be executed by a CPU. In addition, various other peripheral units may be connected to the computer platform such as an additional data storage unit and a printing unit.

It is to be further understood that, because some of the constituent system components and methods depicted in the accompanying drawings are preferably implemented in software, the actual connections between the system components or the process function blocks may differ depending upon the manner in which the present principles are programmed. Given the teachings herein, one of ordinary skill in the pertinent art will be able to contemplate these and similar implementations or configurations of the present principles.

Although the illustrative embodiments have been described herein with reference to the accompanying drawings, it is to be understood that the present principles is not limited to those precise embodiments, and that various changes and modifications may be effected therein by one of ordinary skill in the pertinent art without departing from the scope or spirit of the present principles. All such changes and modifications are intended to be included within the scope of the present principles as set forth in the appended claims.

1. A method comprising:
fastening a sheet of flexible material on a first side of a flexible substrate layer;
adhering a flexible conductive thread to a second side of the flexible substrate;
fastening an electronic component to the substrate and to the conductive path at the location to form an electrical connection of the component to the conductive path; and
applying an adhesive to the second side of the substrate, thereby enabling affixing the substrate including the conductive path and the electronic component to a region of skin of a user's body.

2. The method of claim 1 wherein the sheet of flexible material provides a pattern for fastening the flexible conductive thread to the second side of the flexible substrate.

3. The method of claim 1 further comprising forming the pattern on the sheet of flexible material prior to fastening the sheet of flexible material to the first side of the flexible substrate layer.

4. The method of claim 1 wherein the step of adhering the conductive thread to the second side of the substrate comprises sewing the conductive thread to the second side of the substrate.

5. The method of claim 5 wherein the step of sewing comprises stitching along the pattern using a sewing machine having an upper thread and a lower thread wherein the lower thread comprises the flexible conductive thread, and stitches created by the stitching fasten the sheet of flexible material on the first side of the flexible substrate using the first thread and adhere the flexible conductive thread to the second side of the flexible substrate.

6. The method of claim 5 wherein the upper thread comprises a thread formed from dissolvable PVA material.

7. The method of claim 1 wherein the flexible conductive thread comprises a conductive braided wire.

8. The method of claim 1 wherein the step of applying the adhesive is followed by the steps in the order named of:
adhering the second side of the substrate to the skin of a user using the adhesive,
dissolving the upper thread fastening the flexible sheet of material to the substrate, and
removing the flexible sheet of material from the substrate while leaving the substrate adhered to the skin of the user.

9. The method of claim 1 wherein the sheet of flexible material comprises a transparent PET material and the flexible substrate comprises a silicone material.

10. Apparatus comprising:
a sheet of flexible material;
a flexible substrate layer having a first side to which the sheet of flexible material is fastened;
a flexible conductive thread adhered to a second side of the flexible substrate to form the conductive path, wherein the electronic component is fastened to the flexible substrate layer and to the conductive path at the location to form an electrical connection of the component to the conductive path; and
an adhesive on the second side of the flexible substrate layer configured to enable affixing the flexible substrate layer including the conductive path and the electronic component to a region of skin of a user's body.

11. The apparatus of claim 10 wherein the sheet of flexible material provides a pattern for fastening the flexible conductive thread to the second side of the flexible substrate.

12. The apparatus of claim 10 wherein the flexible conductive thread is adhered to the second side of the flexible substrate layer by sewing the flexible conductive thread to the second side of the flexible substrate layer.

13. The apparatus of claim 12 further comprising a sewing machine stitching along the pattern using a sewing machine having an upper thread and a lower thread wherein the lower thread comprises the flexible conductive thread, and stitches created by the sewing machine fasten the sheet of flexible material on the first side of the flexible substrate layer using the first thread and adhere the flexible conductive thread to the second side of the flexible substrate layer.

14. The apparatus of claim 13 wherein the upper thread comprises a thread formed from dissolvable PVA material.

15. The apparatus of claim 10 wherein the flexible conductive thread comprises a conductive braided wire.

16. The apparatus of claim 10 wherein the adhesive comprises a tattoo paper adhesive.

17. The apparatus of claim 10 wherein the flexible substrate layer is affixed to the skin of a user using the adhesive and thereafter the upper thread is dissolved enabling removal of the flexible sheet of material.

18. The apparatus of claim 10 wherein the sheet of flexible material comprises a transparent PET material and the flexible substrate layer comprises a silicone material.

19. The apparatus of claim 10 wherein the electronic component comprises a sensor affixed to the location on the flexible substrate layer and wherein the substrate including the sensor is configured for attachment to a hand of a user such that the sensor will be positioned on a digit of the hand.

20. The apparatus of claim 10, wherein the apparatus is included in a wearable device, wherein the wearable device includes at least one sensor for sensing a characteristic of the user.
